(19) United States
(12) Patent Application Publication
(10) Pub. No.: US 2003/0124616 A1
(43) Pub. Date: Jul. 3, 2003

(54) Homocysteinylated transthyretin

(76) Inventors: Donald W. Jacobsen, Cleveland, OH (US); Catherine Costello, Reading, MA (US)

Correspondence Address: Raymond A. Miller, c/o Pepper Hamilton LLP, 500 Grant Street, 50thFloor, Pittsburgh, PA, 15219-2502, US

(21) Appl. No.: 10/177,807

(22) Filed: Jun. 21, 2002

Related U.S. Application Data
(60) Provisional application No. 60/299,956, filed on Jun. 21, 2001.

(51) Int. Cl. G01N 33/53; C07K 14/76; C07K 14/47

(52) U.S. Cl. 435/7.1; 530/350; 530/363

(57) ABSTRACT

The present invention is generally directed to the discovery of homocysteinylated proteins and its importance as an indicator of diseased states. In preferred embodiments of the present invention the isolation, detection, and use of homocysteinylated transthyretin are also provided. More preferably, detection and isolation of a homocysteinylated transthyretin complex from a biological fluid is provided in the present invention, as well as methods and compositions utilizing homocysteinylated transthyretin in the detection of homocysteine or elevated levels of homocysteine. More particularly, the present invention is directed to homocysteinylated transthyretin and its use as a marker for the diagnosis of homocysteinemia, hyperhomocysteinemia, and diseases associated therewith.

### CONTINUING DATA

This application claims priority from U.S. Ser. No. 60/299,956, filed Jun. 21, 2001. This application is incorporated herein in its entirety by reference thereto.

### BACKGROUND OF THE INVENTION

Elevated levels of the amino acid homocysteine (Hcy) in plasma and tissue extracts is associated with and/or related to many serious pathological conditions. Homocysteine is a strong independent risk factor of cardiovascular disease, and an elevated blood level of Hcy (i.e., hyperhomocysteinemia or homocystinuria) is a strong predictor of cardiovascular events and death in subjects with preexisting illness. Elevated levels of Hcy are even more pronounced with other compromised organ or medical conditions, including cardiovascular and neurological ailments, for example, heart and kidney transplants, end-stage renal disease, Alzheimer's disease, diabetes, and preclampsia and fetal growth restriction. Hyperhomocysteinemia is a strong independent risk factor for coronary artery disease, cerebrovascular disease, and peripheral vascular occlusive disease. The prognosis for patients with cardiovascular disease (and other diseases as well) combined with high levels of plasma total homocysteine (tHcy) is poor. Recent studies suggest that hyperhomocysteinemia is also a risk factor for other disorders of cognitive dysfunction.

Given the widespread health problems associated with elevated levels of plasma total homocysteine (“tHcy”), it would be advantageous to have an assay for detecting and quantifying Hcy and tHcy to be used in both clinical and research settings.

There are several assays for the determination of tHcy. The existing assays for tHcy include both research and commercial technologies. Current methodologies include HPLC with fluorescence detection, HPLC with electrochemical detection, gas chromatography-mass spectroscopy (GC/MS), tandem mass spectroscopy, fluorescence polarization immunoassay (Abbott Laboratories IMx platform), ELISA (Bio-Rad), chemiluminescence (Diagnostic Products Corporation) and others, as have been previously disclosed and are apparent to those skilled in the art.

### SUMMARY OF THE INVENTION

An embodiment of the present invention provides a composition comprised of homocysteine and a mammalian protein covalently attached in a purified and isolated form. The composition of homocysteine and mammalian protein may be covalently attached by a disulfide bond. The mammalian protein may be selected from the group consisting of albumin, fibronectin, and transthyretin. If the mammalian protein is transthyretin, then Cys₁₀ is involved in forming the disulfide bond.

Another embodiment of the present invention provides a method of determining levels of Hcy in a sample comprising detecting levels of a mammalian protein. The mammalian protein may be selected from the group consisting of albumin, fibronectin, and transthyretin. The levels of Hcy may be indicative of Hcy in the sample. The sample may be a biological sample. Another embodiment of the present invention provides a method of identifying a diseased state comprised of determining the levels of homocysteine in a biological sample from a subject by determining the levels of homocysteinylated mammalian protein in the biological sample. The homocysteinylated mammalian protein may be selected from the group consisting of albumin, fibronectin, and transthyretin. The homocysteinylated transthyretin may be detected using an immunoassay. The immunoassay may be an immunoprecipitation assay. The sample may be a serum sample. The diseased state may be homocysteinemia. In one embodiment of the present invention, an isolated homocysteinylated protein is provided and serves as an indicator of elevated levels of homocysteine and diseased states associated therewith. The homocysteinylated proteins include albumin, fibronectin, and transthyretin.

In a preferred embodiment of the present invention, a novel homocysteinylated transthyretin (Hcy-TTR) complex is provided. Transthyretin (“TTR”) is a 13.8 kDa polypeptide that forms homodimers and homotetramers in circulation. Tetrameric TTR combines with retinol-saturated retinol binding protein for delivery of retinol to cells throughout the body. Tetrameric TTR also carries thyroxin, in circulation. Plasma TTR is a 127 amino acid protein containing a single cysteine residue at position 10. The protein is synthesized in the liver in a constitutive manner, and in circulation exists as a homotetramer. In the present invention, it is disclosed that TTR forms a complex with homocysteine in a dose-dependent manner based on the concentration of plasma tHcy. It is also disclosed that certain other proteins, e.g. fibronectin and albumin, also have a dose-dependent relationship to homeocysteine plasma levels. The identification, purification and isolation of these complexes allows for determination of Hcy in a rapid accurate fashion. Hcy is particularly relevant in this regard.

For convenience, specific reference is made to Hcy-TTR throughout the specification, but it is to be noted that other plasma proteins (e.g., albumin and fibronectin) may find utility in the embodiments described herein. The present invention provides data that demonstrates that Hcy-TTR has been identified in human plasma using immunoprecipitation (IP), high-performance liquid chromatography (HPLC), and electrospray ionization mass spectroscopy (ESIMS) complexed with the aforementioned proteins, including TTR.

Advantageously, the determination of the concentration of homocysteinylated-TTR (Hcy-TTR) serves as an indicator of homocysteine burden in the disease state hyperhomocysteinemia. Accordingly, the determination of Hcy-TTR by IP, HPLC and ESIMS, or by any other methodology such as immunoassay, will serve as a more accurate and specific assay for the diagnosis of hyperhomocysteinemia. While primarily identified as a marker assay, or diagnostic of homocysteine in a biological fluid, the Hcy-TTR complex itself may be useful as a therapeutic agent.

Another embodiment of the present invention provides for a method of detecting the levels of homocysteine present in plasma, serum or other biological sample taken from a subject. The method of detecting Hcy levels is accomplished by measuring the level of Hcy-TTR in the sample, with such measurement being indicative of the Hcy level present in the sample.

Another embodiment of the present invention provides for the method of identifying a subject's diseased state by measuring the level of homocysteine present in a subject's plasma, serum or other biological sample. The level of homocysteine is determined by measuring the level of Hcy-TTR or other homocysteinylated protein present in the sample. The level of Hcy-TTR is measured using an immunoassay, such as an immunoprecipitation assay.

Yet another embodiment of the present invention provides for a method of diagnosis of homocysteinemia. A sample of plasma, serum or other biological sample is contacted with a sample containing transthyretin under conditions such that a specific antigen-antibody binding can occur. The contacting of transthyretin with a subject's sample creates a Hcy-TTR complex. Detection of immunospecific binding of autoantibodies to the Hcy-TTR complex indicates the degree that homocysteine is present in the subject's sample. Detection of the autoantibodies includes the use of a signal-generating component bound to an antibody that is specific for the antibodies in the subject's sample. The presence of autoantibodies in the subject's sample is measured by an immunoassay, which includes the steps of immobilizing homocysteine onto a membrane, contacting the membrane with transthyretin and detecting the presence of autoantibodies specific for the resulting Hcy-TTR complex. Again, the presence of autoantibodies to the Hcy-TTR complex indicates the degree to which homocysteine is present in the subject's sample.

Another embodiment of the present invention provides for pre-packaged diagnostic kits which will be conveniently used in clinical settings, to diagnose or monitor the level of homocysteine in a subject by detecting the level of homocysteine (e.g., via Hcy-TTR) in the subject's serum, plasma or biological sample. The component for detecting Hcy-TTR is an anti-Hcy-TTR antibody, with the anti-Hcy-TTR antibody is labeled. The label is a radioactive, fluorescent, colorimetric or enzyme label. The kit additionally may have a labeled second antibody that immunospecifically binds to the anti-Hcy-TTR antibody.

Another embodiment of the present invention provides for a kit for diagnosis and prognosis of homocysteine levels in a subject, which includes a component for detecting the presence of Hcy-TTR autoantibodies in a subject's sample. The component that is used for detection is an Hcy-TTR antigen that is labeled. The Hcy-TTR antigen is linked to a solid phase. The component is also used to detect the Hcy-TTR autoantibody.

Additional aspects and applications of the present invention will become apparent to the skilled artisan upon consideration of the detailed description of the invention, which follows.

### BRIEF DESCRIPTION OF THE FIGURES

FIG. 1 illustrates in vivo labeling of human plasma (lane 1) and commercially available purified human TTR (lane 2) with ³⁵S-L-homocysteine followed by resolution of labeled protein using SDS-PAGE. Lane 1 shows ³⁵S-TTR (tetramer), albumin, and TTR (dimer) and other higher molecular weight proteins. Lane 2 shows ³⁵S-labeled TTR; predominantly the dimeric form in a purified commercial preparation. To demonstrate L-homocysteine was bound to plasma proteins and purified TTR by disulfide bonds, the labeled plasma sample (lane 3) and labeled TTR sample (Land 4) was treated with β-mercaptoethanol (BME) to break the disulfide bonds and then run on SDS-PAGE;

FIG. 2 illustrates the mass spectrum for the in vitro dose-dependent study for concentration of μM L-homocysteine;

FIG. 3A illustrates the mass spectrum for the in vitro dose-dependent study for concentration of 250 μM L-homocysteine with the charge envelope of intact TTR (16+ charge state is shown as inset);

FIG. 3B illustrates the mass spectrum of the deconvolved mass of intact TTR;

FIG. 4 illustrates in vitro ratio peak intensities (deconvolved) ratios of homocysteinylated TTR (atomic mass of 13,895) to S-Cysteinylated TTR (S-Cys) (Atomic Mass of 13,881).

FIG. 5A illustrates the mass spectrum of the charge envelope for intact protein for the in vivo concentration of 20.7 μM L-homocysteine by the clinical method TTR (16+ charge state is shown as inset);

FIG. 5B illustrates the mass spectrum deconvolved for the in vivo concentration of 20.7 μM L-homocysteine by the clinical method;

FIG. 6A illustrates the mass spectrum of the charge envelope for intact protein for the in vivo concentration of 434 μM L-homocysteine by the clinical method TTR (16+ charge state is shown as inset);

FIG. 6B illustrates the mass spectrum deconvolved for the in vivo concentration of 434 μM L-homocysteine by the clinical method; and

FIG. 7 illustrates the in vivo ratio peak intensities (deconvolved) of S-HomoCys to Scys TTR.

### DETAILED DESCRIPTION OF THE PREFERRED EMBODIMENTS

Before the various embodiments of the present invention are described, it is to be understood that this invention is not limited to the particular methodology, and protocols described, as these may vary. It is also to be understood that the terminology used herein is for the purpose of describing particular embodiments only, and is not intended to limit the scope of the present invention which will be limited only by the appended claims.

It must be noted that as used herein and in the appended claims, the singular forms “a”, “an”, and “the” include plural reference unless the context clearly dictates otherwise. Thus, for example, reference to the “antibody” is a reference to one or more antibodies and equivalents thereof known to those skilled in the art, and so forth.

By “sample” it is meant a volume of fluid or tissue, such as serum, and other bodily fluids, but particularly plasma which is obtained at one point in time. The analyses should be carried out within some short time frame after the sample is taken, as at room temperature Hcy concentrations tend to increase over time due to the protracted production and release of Hcy by blood cells. This process is slowed down when the blood samples are left on ice. While the description particularly identifies TTR, the methods may be used to determine the identity of other plasma proteins, such as fibronectin, that may bind to homocysteine.

The assay devices used according to the invention can be arranged to provide a semiquantitative or a quantitative result. By the term “semiquantitative” is meant the ability to discriminate between a level which is above the elevated marker protein value, and a level which is not above that threshold.

Unless defined otherwise, all technical and scientific terms used herein have the same meanings as commonly understood by one of ordinary skill in the art to which this invention belongs. Although any methods and materials similar or equivalent to those described herein can be used in the practice or testing of the present invention, the preferred methods, devices, and materials are now described. All publications mentioned herein are incorporated herein by reference. Nothing herein is to be construed as an admission that the invention is not entitled to antedate such disclosure by virtue of prior invention.

Positive identification and isolation of homocysteinylated proteins (e.g., Hcy-TTR) is important in its own right and has practical applicability in the diagnosis and treatment of diseases associated with homocysteinemia and in the development of novel assays for the measurement of Hcy levels for the diagnosis of hyperhomocysteinemia and staging of related diseases.

Currently, the clinical diagnosis of hyperhomocysteinemia is dependent upon the determination of tHcy. Although there are a number competing technologies to measure tHcy, there is as yet no reference method for its determination. The ability to measure Hcy-TTR will offer an accurate, specific, and sensitive assay for the diagnosis of hyperhomocysteinemia.

Although not wishing to be bound by theory, it appears most of the Hcy in plasma is protein-bound as disulfide and thus must be released by reduction prior to analysis. Cys₁₀ of TTR appears to form a disulfide bond with homocysteine in a dose-dependent manner based on the concentration of plasma total homocysteine (tHcy). The Cys₁₀ residue is known to carry cysteine, cysteinylglycine, glutathione and sulfite, however, there is no report of this protein carrying homocysteine.

Assays for the determination of tHcy are known in the art. The existing assays for tHcy include both research and commercial technologies. Current methodologies include HPLC with fluorescence detection, HPLC with electrochemical detection, gas chromatography-mass spectroscopy (GC/MS), tandem mass spectroscopy, fluorescence polarization immunoassay (Abbott Laboratories IMx platform), ELISA (Bio-Rad), chemiluminescence (Diagnostic Products Corporation) and others. However, no assays for the determination of Hcy-TTR exist.

Mass spectroscopy was utilized in the present invention. The atomic mass units for the various forms of TTR discussed herein are as follows:

13,761=TTR (parent compound; no conjugation at TTR-Cys₁₀-SH)

13,842=TTR-Cys₁₀-S-S03H (sulfonate) (the reaction product between TTR and sulfite)

13,881=TTR-Cys₁₀-S-S-Cysteine

13,895=TTR-Cys₁₀-S-S-Hcy

13,938=TTR-Cys₁₀-S-S-CysGly

14,068=TTR-Cys₁₀-S-S-Glutathione

In accordance with the present invention, measurement of levels of Hcy-TTR in plasma, serum or body fluid can be used for the detection of hyperhomocysteinemia, and the diseases associated therewith, such as cardiovascular and neurological disorders. Moreover, the monitoring of Hcy-TTR levels can be used prognostically to stage the progression of disease.

The detection of Hcy-TTR in a body fluid from a subject can be accomplished by any of a number of methods. These include but are not limited to the use of HPLC, mass spectroscopy, and others. Preferred diagnostic methods for the detection of Hcy-TTR in the serum of a patient can involve, for example, immunoassays wherein Hcy-TTR are detected by their interaction with an Hcy-TTR specific antibody. Antibodies useful in the present invention can be used to quantitatively or qualitatively to detect the presence of Hcy-TTR. In addition, reagents other than antibodies, such as, for example, polypeptides that bind specifically to Hcy-TTR can be used in assays to detect the level of Hcy-TTR expression.

Immunoassays to be used in the practice of the invention include but are not limited to assay systems using techniques such as Western blots, radioimmunoassays, ELISA (enzyme linked immunosorbent assay), “sandwich” immunoassays, immunoprecipitation assays, precipitin reactions, gel diffusion precipitin reactions, immunodiffusion assays, agglutination assays, complement fixation assays, immunoradiometric assays, fluorescent immunoassays, protein A immunoassays, to name but a few.

In a preferred embodiment, a biological sample which may contain Hcy-TTR, such as serum or other biological fluids, is obtained from a subject. Immunoassays for detecting expression of Hcy-TTR typically comprise contacting the biological sample, such as a serum sample derived from a subject, with a monoclonal anti-Hcy-TTR antibody under conditions such that specific antigen-antibody binding can occur, and detecting or measuring the amount of any immunospecific binding by the antibody. In a specific aspect, such binding of antibody, for example, can be used to detect the presence and increased expression of Hcy-TTR wherein the detection of increased expression of Hcy-TTR is an indication of a diseased condition. The levels of Hcy-TTR in a serum sample are compared to norms that have been established.

The antibody, such as a monoclonal antibody, according to the present invention can be produced by immunizing animals with, as an immunogen (e.g., human Hcy-TTR) obtained according to the present invention based on techniques known or widely applicable in the art. Examples of such techniques are found in Milstein et al., Nature, 256: 495 to 497, 1975, etc., the disclosures of which are hereby incorporated by reference.

In one embodiment of the invention, the biological sample, such as a serum sample is brought in contact with a solid phase support or carrier, such as nitrocellulose, for the purpose of immobilizing any Hcy-TTR complex present in the sample. The support is then washed with suitable buffers followed by treatment with detectably labeled Hcy-TTR specific antibody. The solid phase support is then washed with the buffer a second time to remove unbound antibody. The amount of bound antibody on the solid support is then determined according to well known methods. Those skilled in the art will be able to determine optional assay conditions for each determination by employing routine experimentation.

One of the ways in which Hcy-TTR antibodies can be detectably labeled is by linking the antibody to an enzyme, such as for use in an enzyme immunoassay (EIA) (Voller, A.,“The Enzyme Linked Immunosorbent Assay (ELISA)”, 1978, Diagnostic Horizons 2: 1-7, Microbiological Associates Quarterly Publication, Walkersville, Md.; Voller, A., et al., 1978, J. Clin. Pathol. 31: 507-520; Butler, J. E., 1981, Meth. Enzymol. 73: 482-523). The enzyme which is bound to the antibody will react with an appropriate substrate, preferably a chromogenic substrate, in such a manner as to produce a chemical moiety that can be detected, for example, by spectrophotometric, fluorimetric, or by visual means. Enzymes that can be used to detectable label the antibody include, but are not limited to, horseradish peroxidase and alkaline phosphatase. The detection can also be accomplished by colorimetric methods that employ a chromogenic substrate for the enzyme.

Detection of homocysteinylated protein antibodies, specifically Hcy-TTR antibodies may also be accomplished using a variety of other methods. For example, by radioactively labeling the antibodies or antibody fragments, it is possible to detect Hcy-TTR expression through the use of a radioimmunoassay (RIA) (see, for example, Weintraub, B., Principles of Radioimmunoassays, Seventh Training Course on Radioligand Assay Techniques, The Endocrine Society, March 1986). The radioactive isotope can be detected by such means as the use of a gamma counter or a scintillation counter or by autoradiography.

The antibody may also be labeled with a fluorescent compound. Among the most commonly used fluorescent labeling compounds are fluorescein isothiocyanate rhodamine, phycoerythrin and fluorescamine. Likewise, a bioluminescent compound may be used to label the Hcy-TTR antibody. The presence of a bioluminescence protein is determined by detecting the presence of luminescence. Important bioluminescence compounds for purposes of labeling are luciferin, luciferase and aequorin.

Expression levels of Hcy-TTR in biological samples can be analyzed by one or two-dimensional gel electrophoresis. Methods of two-dimensional electrophoresis are known to those skilled in the art. Two dimensional gel electrophonesis combines separatron by charge and separation by molecular weight. Biological samples, such as serum samples, are loaded onto electrophoretic gels for isoelectric focusing separation in the first dimension which separates proteins based on charge. A number of first-dimension gel preparations may be utilized including tube gels for carrier ampholytes-based separations or gels strips for immobilized gradients based separations. After first-dimension separation, proteins are transferred onto the second dimension gel, following an equilibration procedure and separated using SDS PAGE which separates the proteins based on molecular weight. When comparing serum samples derived from different subjects, it is preferable multiple gels are prepared from individual serum samples.

Following separation, the proteins are transferred from the two dimensional gels onto membranes commonly used for Western blotting. The techniques of Western blotting and subsequent visualization of proteins are also well known in the art (Sambrook et al, “Molecular Cloning, A Laboratory Manual”, 2^{nd} Edition, Volume 3, 1989, Cold Spring Harbor). The standard procedures may be used, or the procedures may be modified as known in the art for identification of proteins of particular types, such as highly basic or acidic, or lipid soluble, etc. (See for example, Ausubel, et al., 1989, Current Protocols in Molecular Biology, Green Publishing Associates and Wiley Interscience, N.Y.). Antibodies that bind to the Hcy-TTR are utilized in an incubation step, as in the procedure of Western blot analysis. A second antibody specific for the first antibody is often utilized in the procedure of Western blot analysis to visualize proteins that reacted with the first antibody.

Hcy-TTR can be used as a marker in the clinical diagnosis of hyperhomocysteinemia. The market for homocysteine assays is rapidly expanding. Hcy-TTR has been an excellent marker for hyperhomocysteinemia. It is particularly useful as a diagnostic or assay in human plasma. A practical clinical diagnostic assay will now be developed. An immuno-based assay will be ideally suited for clinical diagnosis. A monoclonal antibody against Hcy-TTR can be used in any number of settings including ELISA and Abbott Laboratories' IMx platform, which uses fluorescence polarization immunoassay technology.

Materials for TTR Studies

³⁵S-L-Homocysteine thiolactone was synthesized from ³⁵S-L-methionine by a slight modification of the method of Hatch et al. (1961) J. Biol. Chem. 236, 1095-1101 and purified as previously described (Sengupta, S. et al., (2001) J. Biol. Chem. 276, 30111-30117 and Sengupta, S. et al., (2001) J Biol. Chem. 276, 46896-46904 incorporated herein by reference). ³⁵S-L-Homocysteine was prepared from ³⁵S-L-homocysteine thiolactone. The thiol content of each preparation of L-homocysteine was determined by the method of Ellman (Ellman, G. L. (1959) Arch. Biochem. Biophys. 82, 70-77). All experiments were conducted with fresh preparations of L-homocysteine and ³⁵S-L-homocysteine. Purified TTR was obtained from Sigma. Human plasma was obtained from healthy donors, subjects with chronic renal failure, and subjects with homocystinuria using approved protocols.

Radiolabeling and SDS-PAGE of Human Plasma and Purified Transthyretin

³⁵S-L-Homocysteine was incubated with 50% human plasma in 0.05 M TES buffer (pH 7.4), or with purified human transthyretin (1 mg/ml in water) for 5 h at 37 C. Protein was then precipitated with 1.5 M perchloric acid and washed three times with 1.5 M perchloric acid. The final pellet was solubilized in SDS-PAGE sample buffer with and without reduction by β-mercaptoethanol. After treatment at 100 C. for 10 min, aliquots were subjected to analysis by SDS-PAGE using a 10% gel. The gel was stained with Coomassie blue, dried and subjected to phosphorimaging to detect ³⁵S- labeled proteins.

Immunoprecipitation of Transthyretin from Human Plasma

TTR was immunoprecipitated from human plasma as described previously in Théberge et al. (1999) Anal. Chem. 71, 452-459. Briefly, 100 μL of plasma was treated with 80 μL of goat anti-human transthyretin antiserum (Diasorin, Stillwater, Minn.) for 12 h at 37 C. The mixture was centrifuged at 14,000 RPM at room temperature for 20 min. After removal of the supernatant, the pellet was washed with 100 μL of water three times and then dried in a Speed Vac concentrator. The TTR-antibody immunoprecipitate pellets were stored at −70 C. until ready for purification by HPLC.

Purification of Human Transthyretin by HPLC

The method of Theberge et al. (1999) Anal. Chem. 71, 452-459 was used to purify TTR prior to mass spectroscopy. Briefly, the TTR-antibody immunoprecipitate pellets were thawed and dissolved in water-acetonitrile-acetic acid (80:10:10 v/v/v) and passed through a Millipore Microcon YM-100 centrifugal filter (100,000 molecular weight cutoff) to remove the antibody. The filtrate was then applied to an analytical Vydac C-4 HPLC column (25×0.46 cm, 5 μm particle size) and eluted at 0.75 mL/min over 30 min using a gradient of 40-85% acetonitrile. TTR-related components eluted between 52-54% acetonitrile. The solvent mixture was removed from the protein using a Speed Vac concentrator.

Mass Spectrometry of Transthyretin and Transthyretin-Cys₁₀ Analogs

Nanospray mass spectra of intact TTR and TTR-Cys₁₀ analogs were obtained in the positive-ion mode using an Applied Biosystems/MDS-SCIEX QSTAR Pulsar quadrupole/orthogonal acceleration time-of-flight (TOF) mass spectrometer. The instrument was calibrated using the [M+2H]²⁺ ion (m/z 879.9704) and [M+4H]⁴⁺ ion (m/z 440.4892) of porcine renin substrate tetradecapeptide. After calibration, this instrument was capable of achieving approximately 10 ppm mass accuracy with a minimum resolution of 9000 (full with half maximum). Typically, 3 μL of a 1 μM solution of TTR in methanol-water-formic acid (50:50:1 v/v/v) was loaded into a nanospray needle. A stainless steel wire (type 304V, 0.127 mm, 30 gauge; Small Parts, Inc.) was inserted into the nanospray needle containing the sample solution. The capillary potential was increased slowly from 0 up to 1.2 kV until a stable ion current was observed. The declustering potential was held at 35 V.

Determination of Plasma Total Homocysteine

The method of Jacobsen et al. (1994) Clin. Chem, 873-881 was used to determine tHcy. Briefly, plasma or serum samples were reduced with sodium borohydride to liberate free reduced homocysteine, cysteine, cysteinylglycine and glutathione, which were then derivatized with monobromobimane. After removal of proteins by perchloric acid precipitation and centrifugation, the thiol-bimane adducts were resolved by HPLC on a reverse-phase column and detected fluorometrically.

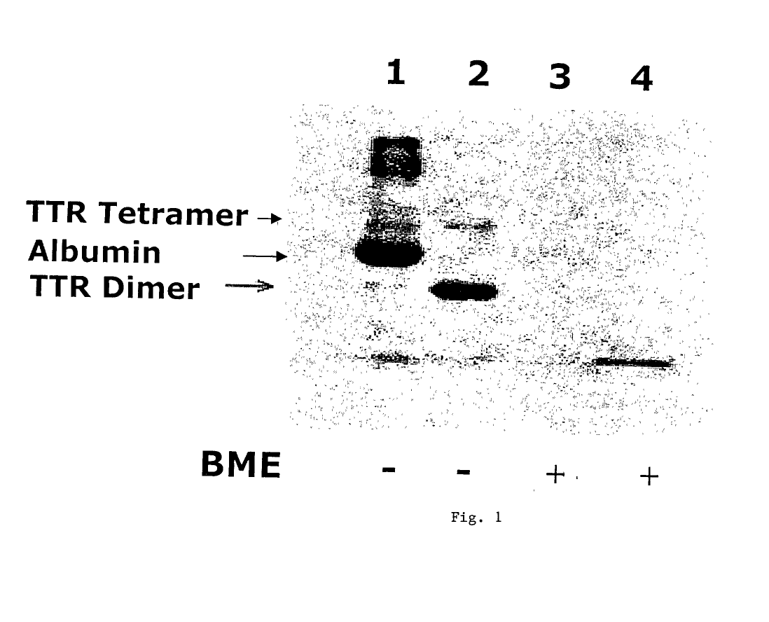

In FIG. 1, ³⁵S-L-Homocysteine (0.5 mM) was incubated with 50% human plasma in 0.05M TES buffer (pH 7.4) and with commercially available human transthyretin from Sigma (1 mg/mL in water) for 5 hours at 35° C. Following the incubation, protein was precipitated with 1.5M perchloric acid and solubilized with SDS-PAGE sample buffer. Aliquots were subjected to analysis by SDS-PAGE using a 10% gel without (see lanes 1 and 2 in FIG. 1) and with reduction by β-mercaptoethanol followed by heating at 100° C. for 10 minutes (see lanes 3 and 4 in FIG. 1). The gel was strained with Coomassie blue, dried and subjected to phosphorimaging to detect ³⁵S-labeled proteins. The objective of this study was to determine if TTR in human plasma reacts with homocysteine under in vitro conditions and if endogenous homocysteinylated TTR is present in normal and hyperhomocysteinemic plasma. Human plasma from healthy donors and purified TTR were incubated with ³⁵S-L-homocysteine for 5 h at 37 C. The samples, before and after treatment with β-mercaptoethanol, were subjected to SDS-PAGE and phosphorimaging. As shown in FIG. 1 (Lane 1), faint bands corresponding to the 27.5 kDa TTR dimer and 55 kDa TTR tetramer were labeled after incubation of normal human plasma with ³⁵S-L-homocysteine. The heavily labeled band in Lane 1 is albumin, which is known to form a disulfide bond with homocysteine. When purified human TTR was incubated with ³⁵S-L-homocysteine (FIG. 1, Lane 2), labeling was found primarily on the dimeric form of TTR. Treatment of plasma or purified TTR with β-mercaptoethanol prior to SDS-PAGE resulted in the loss of the banding patterns for TTR (FIG. 1, Lanes 3 and 4, respectively) suggesting that L-homocysteine was linked to TTR via a disulfide bond. These in vitro experiments provide strong evidence that L-homocysteine readily reacts with the Cys¹⁰ position of TTR.

After treatment with β-mercaptoethanol (FIG. 1, lanes 3 and 4), the loss of the bands on the gel show that β-mercaptoethanol likely broke the disulfide bonds between the ³⁵S-L-homocysteine and TTR.

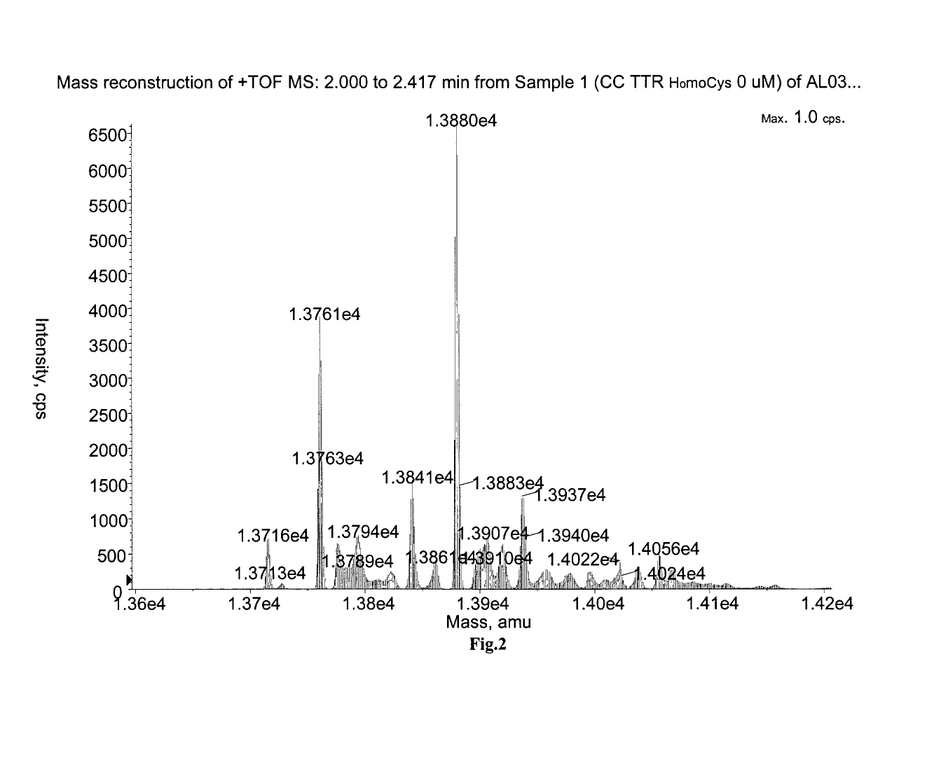

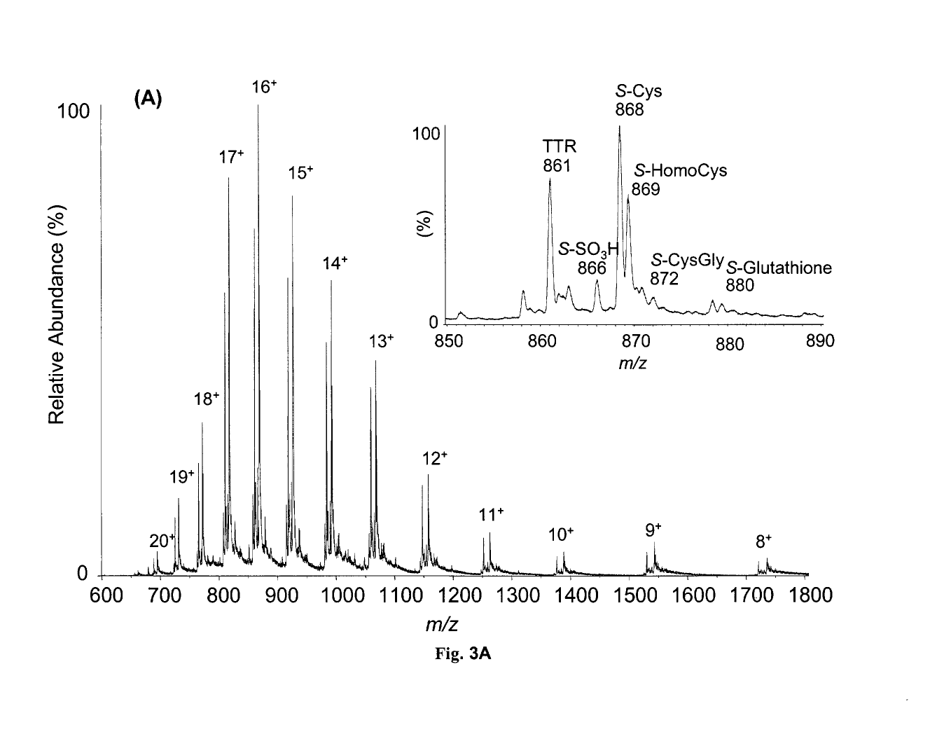

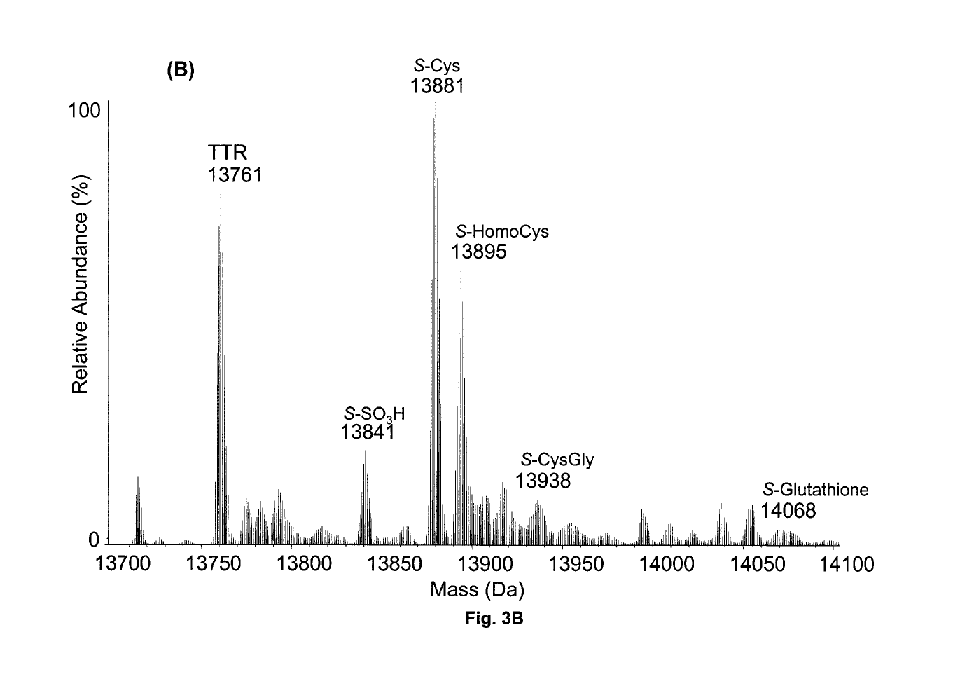

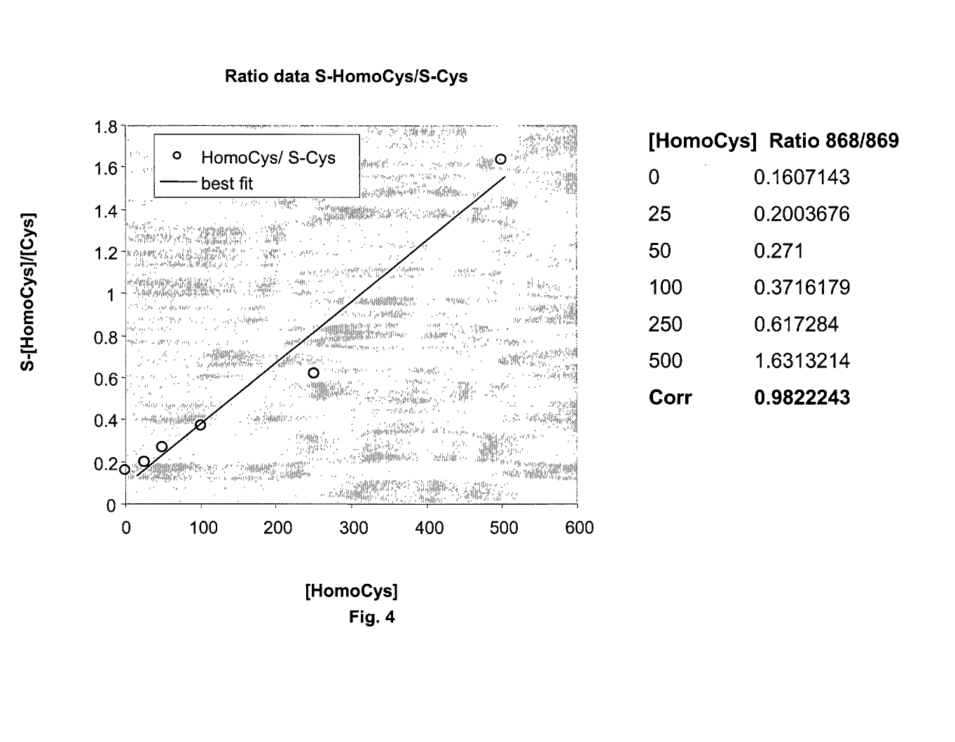

Human plasma from a healthy donor was then treated with increasing concentrations of L-homocysteine (0, 25, 50, 100, 250 and 500 μM) for 5 h at 37 C. TTR was immunoprecipitated and analyzed by HPLC electrospray ionization mass spectrometry (HPLC/ESI/MS). FIG. 2 illustrates the untreated normal plasma (i.e., 0 concentration of Hcy in vitro). The charge envelop of intact TTR for the 250 μM L-homocysteine-treated plasma is shown in FIG. 3A, and the deconvoluted mass of intact TTR is shown in FIG. 3B. In addition to the parent TTR molecule (TTR, mass=13,761), Cys¹⁰ adducts for sulfite (as the S-sulfonate, SO₃-TTR, mass=13,841), cysteine (Cys-TTR, mass=13,881), homocysteine (Hcy-TTR, mass=13,895), cysteinylglycine (CysGly-TTR, mass=13,938) and glutathione (GS-TTR, mass=14,068) are observed. amount of L-homocysteine used in the dose-dependent study, a linear relationship was observed as shown in FIG. 4.

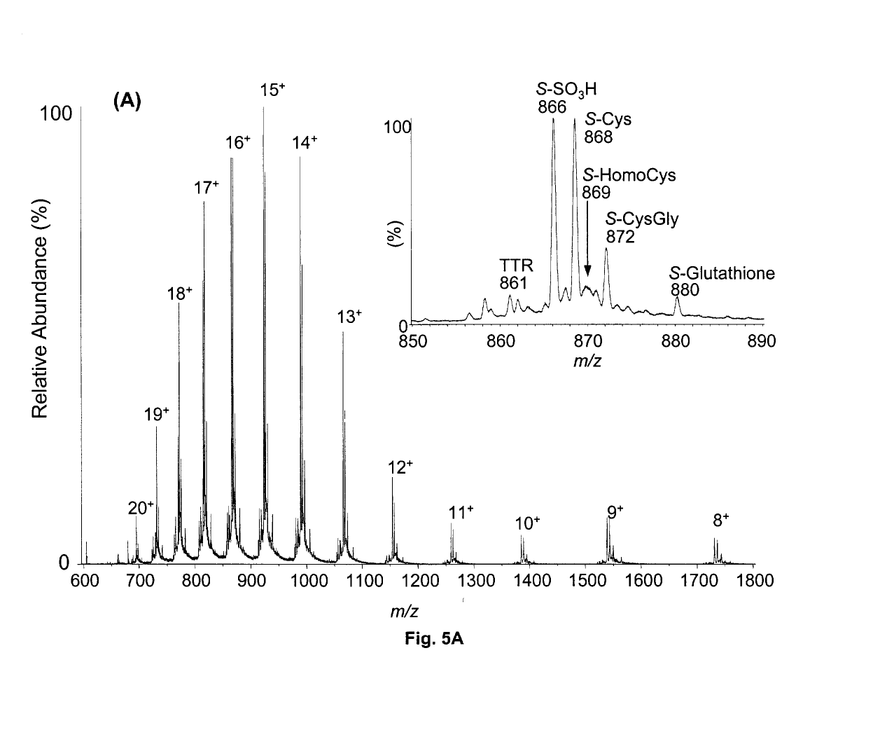

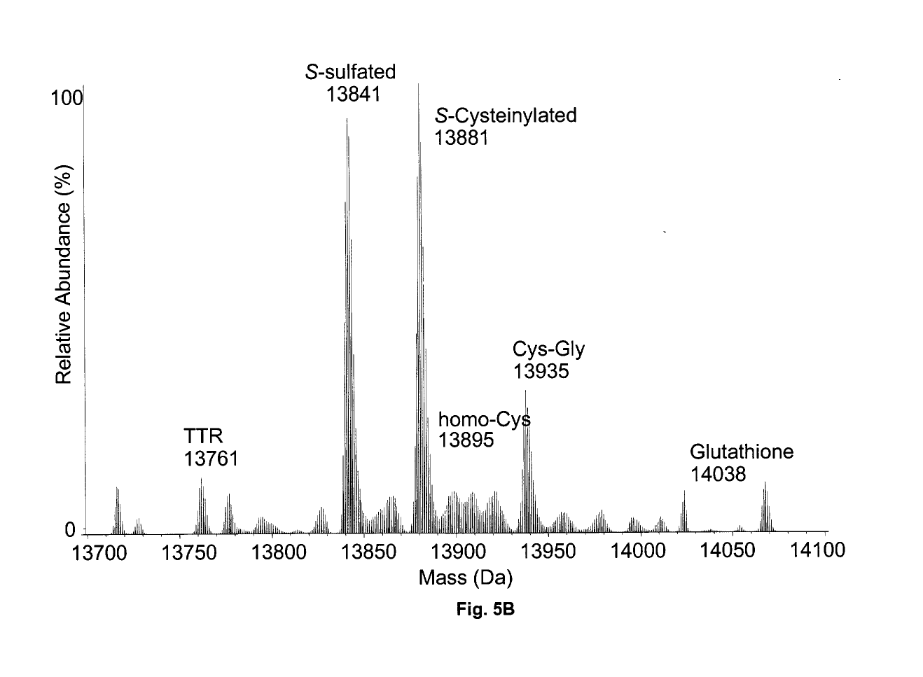

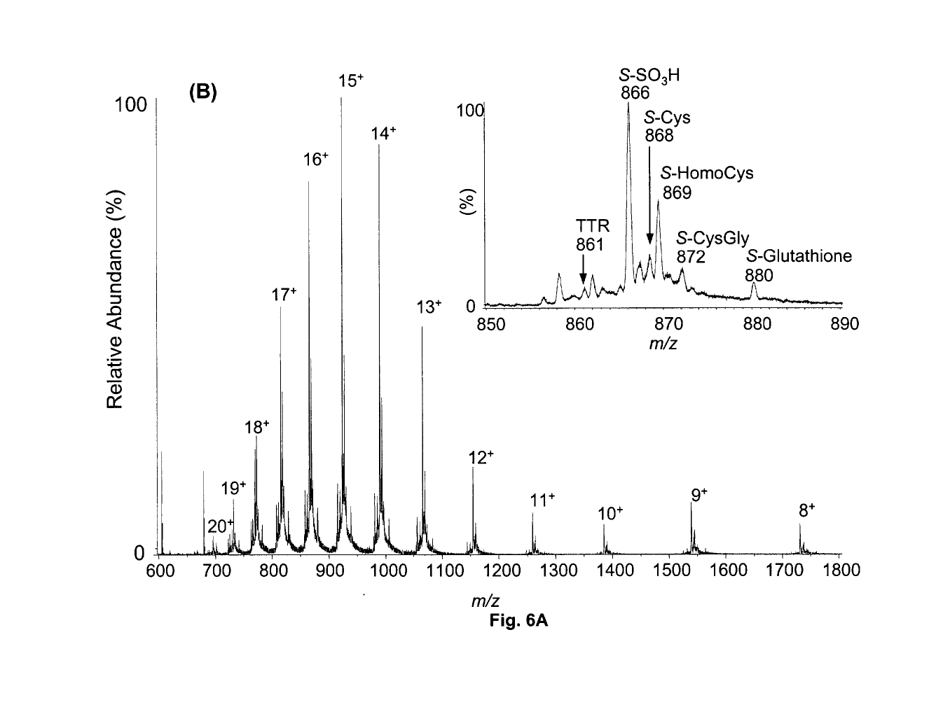

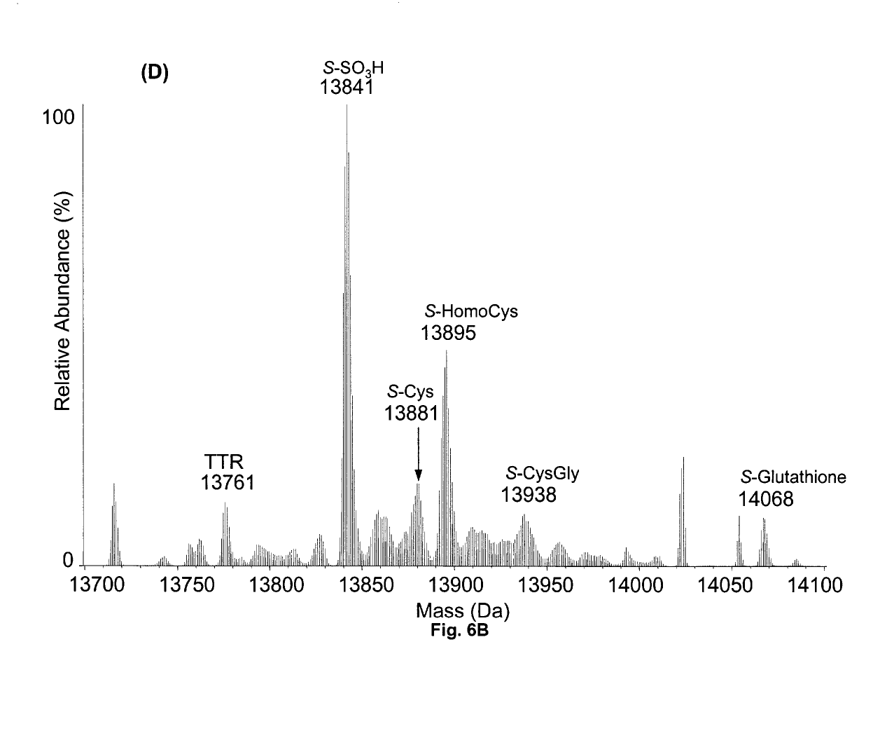

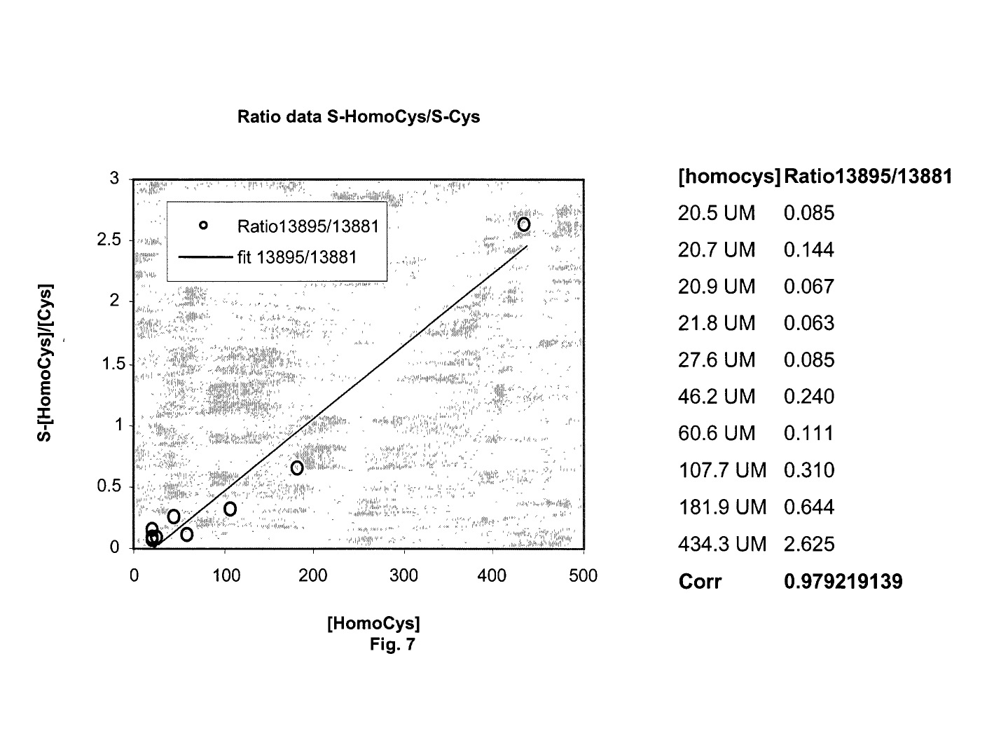

To obtain evidence that TTR reacts with L-homocysteine in human plasma in vivo, plasma from healthy subjects and patients with hyperhomocysteinemia due to chronic renal failure or homocystinuria were examined. Plasma TTR was isolated by immunoprecipitation and analyzed by HPLC/ESI/MS. The charge envelop of intact TTR and the deconvoluted mass of intact TTR isolated from plasma obtained from a patient with end-stage renal disease (tHcy=20.7 μM) is shown in FIGS. 5A and 5B. The major forms of TTR in this plasma sample are TTR-Cys and TTR-SO₃. The charge envelop of intact TTR and the deconvoluted mass of intact TTR isolated from plasma obtained from a patient with homocystinuria (tHcy=434 μM) is shown in FIGS. 6A and 6B. In this plasma SO₃-TTR is the predominant form but Hcy-TTR is now a major adduct, while Cys-TTR is greatly reduced. When the ratio peak intensity of Hcy-TTR over Cys-TTR(n=16⁺ charge state) was compared to the tHcy concentration in normal and hyperhomocysteinemic plasma, a linear relationship was observed (see FIG. 7) suggesting that Hcy-TTR is a direct reflection of homocysteine burden.

The ratios of the peak intensities of the adduct forms of TTR over the parent molecule TTR as function of L-homocysteine concentration in the dose-dependent study are shown in FIG. 6. The ratios for SO₃-TTR, CysGly-TTR and GS-TTR remained relatively constant over the concentration range of L-homocysteine studied. The ratio of Cys-TTR increased dramatically at 500 μM L-homocysteine. This appears to be attributed to a dramatic increase in the concentration of free reduced cysteine from albumin-Cys³⁴-S-S-Cys, which is mediated by L-homocysteine via a thiol/disulfide exchange mechanism.

These studies show that L-homocysteine reacts with TTR in human plasma to form a stable covalent adduct both in vitro and in vivo. This is only the second plasma protein, after albumin, to be identified as a carrier of homocysteine in vivo. A disulfide linkage of homocysteine to TTR is implicated because the Hcy-TTR adduct is sensitive to the reducing agent β-mercaptoethanol. Because TTR contains only one cysteine residue (Cys¹⁰), the Hcy adduct would appear to be TTR-Cys¹⁰-S-S-S-Hcy. Our in vitro and in vivo studies now demonstrate that Hcy-TTR increases in direct proportion to L-homocysteine concentration in the experiment and to the concentration of tHcy in human plasma, respectively.

Methods for Fibronectin

³⁵S-L-Homocysteine Thiolactone Preparation

³⁵S-L-homocysteine thiolactone was prepared from ³⁵S-L-methionine (1,175 Ci/mmol) (New England Nuclear, Boston, Mass.) by the method of Mudd et al.²² with slight modifications. Briefly, unlabeled L-methionine (0.02 mmol) was combined with 1.0 nmol L-³⁵S-methionine and refluxed with hydriodic acid for 18 hours under argon. The resulting solution was dried under argon for 24 hours and resuspended in 0.5 mL water. Descending paper chromatography employing isopropyl alcohol/formic acid/water (70:10:20) was used to separate the homocysteine thiolactone from unreacted methionine. The region corresponding to homocysteine thiolactone was excised and eluted with water. The homocysteine thiolactone was dried in a speed vac and stored at −20° C. At the time of use, an aliquot of ³⁵S-L-homocysteine thiolactone was dissolved in water and the concentration was determined by measuring its absorbance at 243 nm and using a molar absorption of 2.50 M⁻¹ cm⁻¹.

Preparation of L-Homocysteine from L-Homocysteine

Thiolactone Fresh L-homocysteine, both cold and radiolabeled, was prepared from the L-homocysteine thiolactone immediately before use by base hydrolysis as described by Duerre and Miller²³. Briefly, homocysteine thiolactone was hydrolyzed in 5.0 mol/L NaOH for 5 minutes at 37° C., neutralized with 2.0 N HCl and diluted with 50 mmol/L TES (N-tris[hydroxymethyl]methyl-2-aminoethanesulfonic acid) buffer, pH 7.4. Homocysteine concentration was confirmed by reaction with Ellman's reagent, which demonstrated complete conversion of the thiolactone to homocysteine.

Binding of Homocysteine and Cysteine to Plasma Proteins and to Purified Fibronectin

³⁵S-L-homocysteine (500 μmol/L final concentration) was added to either human plasma fibronectin (1 mg/mL, 0.05 mol/L TES buffer, pH 7.4) or human plasma diluted 1:1 with 0.05 mol/L TES buffer, pH 7.4 and incubated under aerobic conditions for 5 hours at 37° C. with continuous shaking. Separate samples of human plasma and fibronectin were incubated with ³⁵S-L-cysteine (1,000 Ci/mmol) (New England Nuclear, Boston, Mass.) which was first diluted with cold L-cysteine (in 1 mol/L TES buffer, pH 7.4) to obtain a specific activity equal to that of the synthesized ³⁵S-L-homocysteine. The final concentrations of L-cysteine and TES in the reaction mixture was 500 μmol/L and 0.05 mol/L respectively and the incubation was carried out as described above for L-homocysteine. The bound radioactive homocysteine was detected by SDS-PAGE of the samples followed by phosphorimage analysis (Molecular Dynamics Inc., Sunnyvale, Calif.).

For fibrin binding, gelatin binding and cell adhesion assays, unlabeled L-homocysteine was bound to fibronectin. Control fibronectin without exogenously added homocysteine was similarly incubated. Unbound homocysteine was removed by precipitating the fibronectin with perchloric acid. Protein content of the resuspended fibronectin with and without bound homocysteine was determined by the Bradford method. Fibronectin was also purified from human EDTA-derived plasma by affinity chromatography on gelatin sepharose followed by heparin sepharose as described by Miekka et al.

Stoichiometry of Homocysteine Binding to Fibronectin

The human plasma fibronectin purified by affinity chromatography described above was used in these studies. Purity was assessed by SDS-PAGE as described below. Purified fibronectin (200 μg/mL) was incubated with various concentrations (up to 500 μmol/L) of ³⁵S-L-homocysteine for up to 10 hours at 37° C. Unbound homocysteine was removed by precipitating the fibronectin with 5% trichloroacetic acid containing 0.5% tannic acid using transferrin as a carrier protein. The precipitated fibronectin was washed 3 times with the trichloroacetic acid/tannic acid, resuspended in 0.1 N NaOH and an aliquot subjected to scintillation counting.

SDS-PAGE

Samples were combined with SDS-PAGE sample buffer (62.5 mmol/L Tris-HCl, pH 6.8, 2% SDS, 10% glycerol, 0.02% bromophenol blue) and aliquots of some samples were reduced with 2-mercaptoethanol and heated to 100° C. for 5 minutes. The samples were loaded onto 4-20% precast gradient gels (Z Axis, Hudson, Ohio) and electrophoresed using the method of Laemmli. Gels were fixed in 50% methanol, 10% acetic acid, stained with Coomassie blue (Gradipure, Pyrmont, Australia), destained with water, dried, and subjected to phosphorimaging.

Limited Tryptic Digestion

³⁵S-L-homocysteine was bound to human plasma fibronectin (Boehringer Mannheim, Indianapolis, Ind.) as described above. An aliquot of this fibronectin solution was precipitated with 1.5 mol/L perchloric acid. The fibronectin was resuspended in TES buffer, the pH adjusted to neutral with 0.1 N NaOH, and subjected to limited trypsin digestion (0.1% trypsin) for 6 hours at room temperature as described. The digestion was stopped by the addition of phenylmethylsulfonylfluoride (PMSF) to a final concentration of 0.1 mmol/L. The digested fibronectin was subjected to SDS-PAGE under nonreducing conditions employing a 4-20% precast gradient gel. The gel was stained with Coomassie blue, dried, and subjected to phosphorimage analysis to identify radiolabeled peptides.

Microsequencing by Liquid Chromatography/Tandem Mass Spectrometry

Three protein fragments containing bound homocysteine (50 kDa, 30 kDa, and 34/35 kDa) that were produced by limited trypsin digestion, were excised from the SDS-PAGE gel and were sequenced. Briefly, the excised bands were destained overnight in 50% methanol/5% acetic acid. The protein was reduced with dithiothreitol and alkylated with iodoacetamide before digestion. The gel bands were washed and the protein digested overnight with trypsin. The resulting peptides were extracted and sequenced using liquid chromatography/tandem mass spectrometry (LC/MS). A Finnigan LCQ-Deca ion trap mass spectrometer system with a Protana microelectrospray ion source interfaced to a self-packed Phenomenex Jupiter C18 reverse phase capillary chromatography column eluted at a flow rate of 0.2 μL/min was used. Peptides were eluted from the column with an acetonitrile/0.05 mol/L acetic acid gradient. Full scan mass spectra were obtained to determine molecular mass of the eluting peptides and product ion spectra were recorded to determine amino acid sequence. Peptides were identified by comparing the peptide sequences obtained to those found in the SwissProt and NCBI databases using the FASTA and SEQUEST database search programs. Amino acid residues are numbered as in SwissProt Feb. 7, 2001, accession number P02751.

Fibronectin Binding to Fibrin

A direct binding assay described by Williams et al.³⁰ was employed to quantify fibronectin binding to fibrin. Human fibrinogen (a generous gift from P. M. DiBello, Cleveland Clinic Foundation) in 0.5 mol/L Tris, 0.1 mol/L NaCl, pH 7.6, was added to 96-well Immulon-2 plates (Dynex Technology, Chantilly, Va.) at 200 ng/well in a total volume of 100 μL and dried to the surface by incubation at 37° C. for 24 hours. Wells were washed with 0.5 mol/L Tris, 0.1 mol/L NaCl, pH 7.6, and a 100 μL mixture of 2 U/mL bovine thrombin (a generous gift from E. Poptic, Cleveland Clinic Foundation) and aprotinin (4 KIU/mL) in 0.5 M Tris, 0.1 mol/L NaCl, pH 7.6 was added and incubated at 37° C. for 2 hours to convert fibrinogen to fibrin. The wells were washed and blocked with 3% bovine serum albumin (BSA) in 0.05 mol/L Tris, 0.1 mol/L NaCl, pH 7.6 for 1 hour at 37° C. Human plasma fibronectin with or without bound homocysteine was then added (200 ng/well) and the plates were incubated for 1 hour at room temperature to allow binding of fibronectin to the fibrin. The fibronectin concentration was predetermined to ensure all wells received equivalent amounts of fibronectin. The bound fibronectin was detected by ELISA employing a monoclonal antibody recognizing the fifth type III repeat (Sigma Chemical Co.), a goat anti-mouse secondary antibody conjugated to horseradish peroxidase (Southern Biotech, Birmingham, Ala.), and the substrate o-phenylenediamine. Optical density at 492 nm was quantified on a Molecular Devices Spectra Max 190 multiwell plate reader. A standard curve was generated by coating wells with various amounts of purified human plasma fibronectin, blocking with 3% BSA, and detecting it as described above.

Cell Adhesion Assay

Cell adhesion was assayed using a method based on those described by Woods et al. and Meighen et al. Control fibronectin, or fibronectin with bound homocysteine was diluted in 0.1 mol/L Tris, pH 9.1 and 1.4 μg per well was added to Immulon II 96 well plates. The plates were incubated overnight at 4° C. The wells were washed with PBS, blocked with 4% BSA in PBS for 1 hour at 37° C. and rinsed with PBS prior to the addition of cells. Control wells contained only the BSA, and no fibronectin. Rabbit aortic smooth muscle cells (SMCs) were cultured in DME/F12 with Hepes buffer (Gibco BRL, Grand Island, N.Y.) that contained 10% fetal bovine serum (BioWhittaker, Walkersville, Md.), and an antibiotic/antimycotic (Gibco BRL) as described. The SMCs were incubated in complete medium containing 25 μg/mL cycloheximide for 2 hours at 37° C. prior to the assay. The cells were harvested with 5.0 mmol/L EDTA in PBS, rinsed in serum free medium containing 25 μg/mL cycloheximide, resuspended in serum-free medium with 25 μg/mL cycloheximide, and plated at a density of 10⁵ SMCs per well. The plates were incubated at 37° C. for 1 hour, and washed with serum-free medium. Attached cells were fixed in 10% formalin, washed with water, and stained for 30 minutes with 0.2% crystal violet in 80% methanol. The wells were washed with water, the stain was solubilized with 0.1 mol/L sodium citrate, pH 4.2, and absorbance at 590 nm was measured.

Gelatin Binding Assay Immulon II

96 well plates were coated overnight with 200 ng per well of gelatin in 0.1 mol/L Tris, pH 9.1. The wells were rinsed with Tris buffered saline (TBS), and blocked with 3% BSA in TBS. Fibronectin with and without bound homocysteine was added and incubated at room temperature for one hour. The wells were washed with TRBS containing 0.1% BSA and 0.1% Tween. Bound fibronectin was quantified by ELISA as described for fibrin binding, except a rabbit anti-human fibronectin antibody (Sigma) and swine anti-rabbit secondary antibody conjugated to horseradish peroxidase (Dako, Carpinteria, Calif.) were employed. A standard curve was generated with fibronectin to ensure the fibronectin bound to gelatin was within the linear range of detection.

Homocysteine and Cysteine Binding to Plasma Proteins and Purified Fibronectin

To identify the plasma proteins that bind homocysteine, normal human plasma was diluted 1:1 with TES buffer and incubated with 500 μmol/L ³⁵S-L-homocysteine followed by separation of the proteins by SDS-PAGE and visualization of the radioactive proteins with. Although most of the radioactivity in the plasma was associated with albumin, one of the other radioactive bands comigrated with purified human plasma fibronectin. Treatment of the plasma and fibronectin samples with 2-mercaptoethanol prior to SDS-PAGE released the radioactive homocysteine, demonstrating that the homocysteine was bound via a disulfide linkage. Human plasma and purified fibronectin were also incubated with 500 μmol/L ³⁵S-L-cysteine. SDS-PAGE analysis and phosphorimaging demonstrate that although the radiolabeled L-cysteine bound to albumin, it did not bind to either the fibronectin in the plasma, or to purified fibronectin.

The binding of homocysteine to fibronectin increased with increasing homocysteine concentration. In the presence of 500 μmol/L homocysteine, the binding of homocysteine to fibronectin reached equilibrium within 5 hours. A maximum of 5 moles of homocysteine were bound per mole of dimeric fibronectin.

Localization of Bound Homocysteine within Fibronectin

To identify the region(s) of fibronectin to which homocysteine binds, an aliquot of fibronectin labeled with ³⁵S-L-homocysteine was subjected to limited trypsin digestion and the tryptic fragments were separated by SDS-PAGE under nonreducing conditions. The radioactive bands were detected with. Several, but not all of the resulting protein fragments were radioactive. Low molecular weight labeled fragments of molecular masses of 50 kDa and 30 kDa, and a doublet at 34/35 kDa were excised from the gel, reduced, alkylated, and subjected to exhaustive trypsin digestion. The resulting peptides were sequenced using LC/MS. The majority of the identified peptides lie within and adjacent to the C-terminal fibrin binding domain.

The 30 kDa band contained 10 peptides. Eight of these peptides were from the C-terminal region, between residues 2150 and 2356, within and adjacent to the fibrin binding domain, and near a free cysteine sulfhydryl group. The remaining 2 peptides were from the N-terminus and contained amino acids 58-67 and 133-140, which are located in the N-terminal fibrin binding domain. The 34/35 kDa doublet contained 17 fibronectin peptides. The eleven most abundant peptides were again located between amino acids 2150 and 2356, within and adjacent to the fibrin binding domain. Five peptides containing 65 amino acids were from residues 1822 to 1910. The last peptide was significantly less abundant and again contained residues 58-67 from the N-terminus. The 50 kDa fragment was present in both the undigested, and the digested fibronectin, and contained 7 fibronectin peptides containing 97 amino acids from near the C-terminus. These peptides represented residues 1286-1301, 1788-1796, 1867-1910, and 2150-2176. Also present in this digest were peptides from bovine serum albumin, a protein known to bind homocysteine, and a contaminant in the commercial fibronectin preparation employed in this experiment.

Effect of Bound Homocysteine on Fibronectin Binding to Fibrin

A direct binding assay was employed to determine if the binding of homocysteine in or near the C-terminal fibrin binding domain affected the interaction of fibronectin with fibrin. Homocysteinylated fibronectin and control fibronectin were prepared by incubating fibronectin with or without 500 μmol/L L-homocysteine for 5 hours at 37° C. The amount of homocysteinylated fibronectin that bound to fibrin was only 38% of that of control fibronectin without bound homocysteine.

LC/MS sequence analysis detected and characterized a relatively extensive series of peptides in each of the three radiolabeled bands. The majority of these peptides were mapped to the C-terminus of the protein sequence, within and adjacent to the C-terminal fibrin-binding domain. This region of the protein also contains a free cysteine residue that may be capable of binding homocysteine. Of the additional peptides that were detected in each of these analyses, several peptides in the 50 kDa band were mapped to albumin, a contaminant in the commercial fibronectin preparation that was used, and a protein known to bind homocysteine. The remaining peptides, detected in the 34/35 kDa band and 30 kDa band, were three fibronectin peptides from an N-terminal region that is also a fibrin-binding domain. The presence of these additional peptides is most likely due to co-migration in the nonreducing SDS-PAGE separation of the limited proteolysis products. Overall, however, the pattern of peptides that were detected in these radiolabeled bands clearly supports the C-terminal region of the fibronectin sequence as a major homocysteine binding domain. Additional experiments were carried out to test the significance of homocysteine binding in this region.

Two fibrin-binding domains present in each subunit mediate the binding of fibronectin to fibrin. The first is located at the N-terminus and is the primary binding site. A second site is located near the C-terminus. This fibrin-binding domain is in a region of fibronectin containing numerous intrachain disulfide bonds. The reduction of fibronectin by dithiothreitol reduced the interaction of fibronectin with fibrin, thereby demonstrating the importance of intact disulfide bonds for efficient interaction. This finding suggested that prolonged exposure of circulating fibronectin to select biological thiols may inhibit its interaction with fibrin in vivo. Our results demonstrate that homocysteinylation of fibronectin significantly impairs its interaction with fibrin. Homocysteine might be breaking crucial disulfide linkages via thiol/disulfide exchange or might be binding to the free cysteine residue near the fibrin binding domain.

In addition to its interaction with fibrin, fibronectin binds to gelatin and serves as an attachment factor for numerous cell types. Homocysteinylated fibronectin was just as effective as control fibronectin as an adhesion protein for aortic SMCs. The presence of bound homocysteine also had little effect on the binding of fibronectin to gelatin. These results suggest that the binding of homocysteine to fibronectin has specific effects, and the major physiologic consequence of homocysteine binding is a reduction in the capacity of fibronectin to bind fibrin.

The binding of fibronectin to fibrin is important in thrombosis and wound healing. Tissue injury produces a rapid induction of the clotting cascade and the formation of a provisional matrix, the major components of which are fibrin and fibronectin. These processes are carefully orchestrated, and dysregulation of the sequential steps involved in their progression impairs normal blood clotting and wound healing. As wound healing progresses, the fibrin/fibronectin-rich clot is replaced by a matrix rich in fibroblasts and blood vessels, and finally by a collagen-rich scar. The provisional matrix serves as a substrate for the adhesion and migration of mesenchymal cells. Fibroblasts and endothelial cells migrate from the periwound area to the provisional matrix as part of the transformation to granulation tissue. Fibronectin is primarily responsible for the adhesion, spreading, and migration of the invading cells. The fibronectin also significantly increases the retraction of the clot by nucleated cells.

Inhibition of the binding of fibronectin to fibrin resulting in a reduction of the incorporation of fibronectin into the provisional clot may impair the early events in wound healing and tissue remodeling. Low levels of plasma fibronectin present in individuals suffering from starvation, shock, burns, trauma, infection and certain metabolic diseases may impair wound healing. Malnourished rats with low plasma fibronectin have delayed wound healing. However, the intravenous administration of fibronectin to malnourished rats significantly improved wound healing, demonstrating the importance of plasma fibronectin. The impairment of fibrin binding by bound homocysteine may result in a deficiency of fibronectin in the provisional clot and subsequent delays in wound healing and clot resolution.

The formation of thrombi on atherosclerotic lesions can lead to occlusion, ischemia, and death. A significant correlation between total plasma homocysteine levels and the rate of cardiovascular disease mortality may exist. A strong relationship between total homocysteine levels and mortality due to cardiovascular events may also exist. When all causes of death were included, the relationship was still strong. About 24.7% of patients with total homocysteine levels above 15 μmol/L had died after 4 years, as compared to only 3.8% for patients with total homocysteine levels below 9 μmoles/L. Homocysteine levels may be strongly related to acute events resulting in death. Most acute ischemic cardiac syndromes such as myocardial infarction, unstable angina, and sudden cardiac death, are the result of atherosclerotic plaque rupture and subsequent thrombosis. A delay in the resolution of the clot may lead to the presence of a prolonged thrombogenic site, which may serve as a nidus for further platelet accumulation and clot formation.

Atherosclerosis and restenosis have long been viewed as a form of prolonged wound healing. The spatial temporal pattern of events following angioplasty, including deposition of extracellular matrix is quite analogues to healing wounds, and culminate in lumen narrowing. In addition to the physical wounds caused by angioplasty, plaque fractures and plaque ruptures can also be viewed as “wounds”, which may eventually result in lumen narrowing as a result of normal, prolonged wound healing. A failure of vascular smooth muscle cells to repair wounds adequately may play a part in plaque rupture and sudden death. Fibronectin present in the clot links fibrin and smooth muscle cells and demonstrated that plasma protease inhibitors are required to prevent degradation of fibronectin. An inhibition of fibronectin binding to fibrin by homocysteine may lead to a deficiency of fibronectin in the clot. Such a deficiency of fibronectin may thus contribute, in part, to inadequate wound repair, plaque rupture and sudden death.

An alternative embodiment of the present invention is a utilization of the basic discoveries presented herein in the creation of a ratiometric analysis of the amount of homocysteine in biological fluids compared to levels of cysteine. This is shown in the figures regarding Hcy-TTR but is applicable to the other proteins disclosed herein. The levels of Hcy-TTR will be compared to the levels of cysteinylated-TTR in the biological fluid to indicate a diseased state. This embodiment will be particularly useful in those conditions in which homocysteine levels are decreased due to malnutrition or end-stage disease state. Additionally, it would appear that other homocysteinylated proteins within a biological fluid may be utilized to measure homocysteine levels in accordance with the methods, analysis, kits and procedures set-forth herein. For example, albumin which contains a cysteine residue would be expected to be a carrier of homocysteine; therefore, proteins with exposed cysteine residues that exist in a biological fluid may be utilized in accordance with the present invention to indicate the levels of homocysteine or a hypo-homocysteinylated state.

Based on the dose-dependent studies above, a number of embodiments can be envisioned. Kits, for example, would be particularly advantageous. The present invention further provides for kits for carrying out the above-described assays. The assays described herein can be performed, for example, by utilizing pre-packaged diagnostic kits, comprising at least an Hcy-TTR peptide (for detection of Hcy-TTR autoantibodies) or an Hcy-TTR antibody reagent (for detection of Hcy-TTR), which can be conveniently used, e. g., in clinical settings to diagnose the level of Hcy.

In a first series of nonlimiting embodiments, a kit according to the invention comprises components for detecting and/or measuring human IgG antibodies directed toward Hcy-TTR antigen. As one example, where the antibodies are detected and/or measured by enzyme linked immunoabsorbent assay (ELISA), such components may comprise target antigen, in the form of at least one and preferably a plurality of different Hcy-TTR antigens or epitopes thereof, linked to a solid phase, and a means for detecting a human antibody antibody bound to target antigen. Such means for detection may be, for example, an antibody directed toward the constant region of human IgG (e. g., rabbit anti-human IgG antibody), which may itself be detectably labeled (e.g., with a radioactive, fluorescent, calorimetric or enzyme label), or which may be detected by a labeled secondary antibody (e.g., goat anti-rabbit antibody).

In a second series of nonlimiting embodiments, a kit according to the invention may comprise components which detect and/or measure Hcy-TTR antigens in the biological sample of a subject. For example, where Hcy-TTR is detected and/or measured by enzyme linked immunoabsorbent assay (ELISA), such components may comprise an antibody directed to epitopes of the Hcy-TTR which can be used to detect and/or quantitate the level of Hcy-TTR expression in the biological sample. The antibody itself may be detectably labeled with a radioactive, flourescent, calorimetric or enzyme label. Alternatively, the kit may contain a labeled secondary antibody.

All of the embodiments disclosed and claimed herein can be made and executed without undue experimentation in light of the present disclosure. While the compositions and methods of this invention have been described in terms of preferred embodiments, it will be apparent to those of skill in the art that variations may be applied to the composition, methods and in the steps or in the sequence of steps of the method described herein without departing from the concept, spirit and scope of the invention. More specifically, it will be apparent that certain agents that are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

What is claimed is:

1. A composition comprised of homocysteine and a mammalian protein covalently attached in a purified and isolated form.

2. The composition of claim 1, wherein said homocysteine and said mammalian protein are covalently attached by a disulfide bond.

3. The composition of claim 1, wherein said mammalian protein is selected from the group consisting of albumin, fibronectin, and transthyretin.

4. The composition of claim 1, wherein said mammalian protein is transthyretin.

5. The composition of claim 4, wherein Cys₁₀ is involved in forming the disulfide bond.

6. A method of determining levels of Hcy in a sample comprising detecting levels of a mammalian protein.

7. The method of claim 6, wherein said mammalian protein is selected from the group consisting of albumin, fibronectin, and transthyretin.

8. The method of claim 6, wherein said mammalian protein is transthyretin.

9. The method of claim 6, wherein said levels of Hcy are indicative of Hcy in said sample.

10. The method of claim 6, wherein said sample is a biological sample.

11. A method of identifying a diseased state comprised of determining the levels of homocysteine in a biological sample from a subject by determining the levels of homocysteinylated mammalian protein in the biological sample.

12. The method of claim 11, wherein said homocysteinylated mammalian protein is selected from the group consisting of albumin, fibronectin, and transthyretin.

13. The method of claim 11, wherein said homocysteinylated mammalian protein is transthyretin.

14. The method of claim 13, wherein said homocysteinylated transthyretin is detected using an immunoassay.

15. The method of claim 14, wherein said immunoassay is an immunoprecipitation assay.

16. The method of claim 11, wherein the sample is a serum sample.

17. The method of claim 16, wherein the diseased state is homocysteinemia.

18. The method of claim 11 further comprising:
contacting the serum sample with a sample containing homocysteinylated protein, thereby creating a Hcy-protein complex;
further contacting said Hcy-protein complex with an antibody under conditions such that a specific antigen-antibody binding can occur; and
detecting immunospecific binding of the antibody to the Hcy-protein complex, to thereby determine the amount of homocysteine present in the serum sample.

19. The method of claim 18, wherein said antibody is a monoclonal antibody.

20. The method of claim 19, wherein the step of detecting the antibodies in the subject's serum sample comprises the use of a signal-generating component bound to an antibody that is specific for antibodies to the homocysteinylated mammalian protein in the subject's serum sample.
